(19) United States
(12) Patent Application Publication
(10) Pub. No.: US 2002/0055539 A1
(43) Pub. Date: May 9, 2002

(54) Compositions and methods for treating cardiovascular conditions

(76) Inventors: Barry I. Bockow, Seattle, WA (US); Marc D. Erlitz, Kirkland, WA (US); Philip J. Mease, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/814,394

(22) Filed: Mar. 21, 2001

Related U.S. Application Data
(63) Continuation of application No. 09/517,421, filed on Mar. 2, 2000, now abandoned.
Application No. 09/517,421 is a continuation of application No. 09/189,438, filed on Nov. 10, 1998, now abandoned.
Application No. 09/189,438 is a continuation of application No. 08/725,072, filed on Oct. 2, 1996, now abandoned.

(51) Int. Cl. A61K 31/202

(52) U.S. Cl. 514/560

(57) ABSTRACT

There is disclosed compositions and methods for treating or preventing cardiovascular conditions by intravascular administration of an omega fatty acid to a patient in need thereof. The omega fatty acid is intravascularly administered preferably in close proximity to the treatment site. Cardiovascular conditions which may be treated or prevented according to this invention include coronary artery disease, myocardial infarction, cerebrovascular disease, stroke, peripheral vascular disease, and atherosclerosis or thrombosis of arteries or veins supplying any organ system. Thrombosis or restenosis occurring in grafts, stents, and in areas of diagnostic or therapeutic intervention such as angioplasty or diagnostic radiology sites can also be treated or prevented.

### CROSS-REFERENCE TO PRIOR APPLICATION

This application is a continuation of U.S. patent application Ser. No. 08/725,072, filed Oct. 2, 1996, now pending.

### TECHNICAL FIELD

The present invention relates generally to compositions and methods for treating cardiovascular conditions by intravascular administration of an omega fatty acid.

### BACKGROUND OF THE INVENTION

Arteriosclerotic cardiovascular disease (ASCVD) is one of the leading causes of morbidity and mortality in both America and the world. Coronary artery disease, myocardial infarction, cerebral vascular disease, stroke, and peripheral vascular disease are all manifestations of the arteriosclerotic process.

There are a number of methods currently used to treat ASCVD and its complications. Primary prevention methods include reduction of cholesterol, smoking avoidance, normalizing blood pressure, decreasing weight and increasing exercise. If ASCVD progresses to the point of complete occlusion of an artery by thrombus formation, grave consequences can occur due to loss of blood flow to part of the body (ischemia). If a coronary artery is blocked, various complications arise depending on the location of the artery. These include chest pain from myocardial infarction or heart muscle ischemia (angina), dangerous irregularities of heart rhythm, clot formation within heart chambers which can embolize to the brain, and heart failure, characterized by fluid accumulation in the lungs, respiratory failure, low blood pressure and inadequate flow of blood to vital organs. These complications lead to a high rate of morbidity and mortality. ACSVD-induced blockage of arteries to other major organs such as the brain, kidney and bowel cause selective organ failure and sometimes death.

When coronary arteries become blocked, leading to myocardial infarction (MI) or “heart attack,” conventional therapies involve stabilizing the patient's blood pressure, treating arrhythmias and attending to associated failure of other organs. Cell death due to ischemia is reduced by attempting to re-open blood flow through the blocked artery and by other supportive measures. Current methods of reperfusion include thrombolytic therapy, e.g., infusion of medicines to open blocked arteries, dilating the blocked vessel by inflating a small balloon in the occluded area via percutaneous transluminal coronary angioplasty (PTCA), and surgical bypass of the blocked artery (coronary artery bypass graft). While intravenous infusion of medicines such as streptokinase or tissue plasminogen activator (TPA) may help to open blocked arteries, such medicines have limited effectiveness, a high rate of restenosis, and can be complicated by serious bleeding, especially in the brain.

PTCA is performed on more than 400,000 Americans annually, and involves the use of a sophisticated catheterization lab. Unfortunately, restenosis occurs in approximately 40% of patients within six months, requiring repetition of the procedure or coronary artery bypass surgery. The effectiveness of PTCA is considerably less in the acute MI setting if there is a delay in transport of the patient, diagnosis, and/or initiation of the procedure. A similar problem occurs in relation to bypass surgery, which can also be quite risky with significant morbidity and 2-4% mortality. Finally, all of these procedures are very costly, e.g., the cost of open heart surgery can exceed $50,000 per patient. Clearly, effective strategies to prevent or treat ASCVD in general, as well as specific coronary conditions such as restenosis, are needed.

One such strategy is dietary supplementation with omega fatty acids. Epidemiological studies have implicated an inverse relation between mortality from coronary heart disease and the dietary intake of omega-3 fatty acids derived from marine vertebrates. Beginning in 1987, multiple clinical trials have been mounted to test the efficacy of dietary supplementation with omega-3 fatty acids for the treatment and prevention of atherosclerosis and restenosis following PTCA. Moreover, there has been extensive discussion in the literature concerning the effects of long-term dietary supplementation with polyunsaturated fatty acids, particularly omega-3 fatty acids found in fish oil, on the favorable modulation of arteriosclerosis and cardiovascular disease risk factors. However, in spite of this tremendous volume of work, oral administration of omega-3 fatty acids has not been demonstrated to be so dramatic in its effectiveness as to lead to its widespread use (Endres, S. et al., Eur. J. Clin. Invest. 25:629-638, 1995). This situation is compounded by side effects associated with oral administration, particularly with regard to gastrointestinal intolerance.

As of June 1994, there were nine reported clinical trials documenting the effect of fish oil supplementation on the course of restenosis following PTCA. Two meta-analyses (O'Connor, G. T. et al., Am J. Prev. Med. 8:186-192, 1992; and Gapinski, J. P. et al., Arch. Intern. Med. 153:1595-601, 1993), analyzing the data from the first seven studies, concluded that while the combined data were compatible with small to moderate benefit of fish oil on the prevention of restenosis, larger studies were needed to reliably distinguish meaningful benefit from null results. This conclusion has now been put in perspective by the Fish Oil Restenosis Trial of Leaf, A. et al. (Circulation 90:2248-57, 1994). With 512 patients, this is the largest clinical trial to date. Dietary supplementation, from 12 to 14 days before through 6 months after angioplasty, with 8 g/d of omega-3 fatty acids failed to prevent the usual high rate of restenosis after PTCA; 46% in the corn oil and 52% in the fish oil group (P=0.37). Yet another study of 212 patients by Franzen, D. et al. (Cathet. Cardiovas. Diag. 28:301-310, 1993), failed to demonstrate any effect of omega-3 fatty acid dietary supplementation on coronary artery restenosis.

The explanation for these differing clinical results is not apparent. The numbers of patients were generally small, procedures varied, dosage and form (ethyl ester vs. triglyceride) varied, as did the time of supplement commencement in relation to PTCA. Moreover, depending on exclusion criteria, in some trials patients were treated with other medications prescribed by their physician. Given that omega-3 fatty acids affect a diverse set of biological processes, simultaneous administration with other drugs might unpredictably modify trial results. Alternatively, response differences may be in some way related to the overall dietary proportions of poly- and mono-unsaturated fatty acids with that of saturated fatty acids (i.e., the P/M/S ratio). For example, in a study involving swine on atherogenic diets (Whitman, S. C. et al., Arterioscler. Thromb. 14(7):1170-76, 1994), supplementation with fish oil failed to show effects on atherosclerotic lesion formation when the P/M/S ratio was held constant between omega-3 fatty acid supplemented and control groups.

Despite the advances made in this field, more efficacious and cost effective therapies are needed for the general treatment of occlusive vascular conditions, particularly for the prevention of morbidity and mortality associated with restenosis following angioplasty and myocardial infarction. The present invention fulfills these needs and provides further related advantages.

### SUMMARY OF THE INVENTION

Briefly stated, the present invention discloses methods and compositions for treating a cardiovascular condition by intravascular administration of an omega fatty acid to a patient in need thereof. In the practice of this invention, the omega fatty acid is intravascularly administered locally at or in close proximity to the site of occlusion (also referred to herein as the “treatment site”). Cardiovascular conditions which may be treated according to this invention include coronary artery disease, myocardial infarction, cerebral vascular disease, stroke, peripheral vascular disease and atherosclerosis or thrombosis of arteries or veins supplying an organ system. Thrombosis or restenosis can also occur as a result of angioplasty, or in association with grafts or stents.

In one embodiment, the omega fatty acid is an omega-3 fatty acid selected from eicosapentaenoic acid and docosahexaenoic acid. In a further embodiment, the omega fatty acid is an omega-6 fatty acid, or a combination thereof with an omega-3 fatty acid. Compositions containing an omega fatty acid in combination with a pharmaceutically acceptable carrier or diluent are also disclosed.

In another embodiment, a method for preventing or delaying restenosis is disclosed, such as restenosis following angioplasty, by local intravascular administration of an omega fatty acid at the site of the angioplasty. In further embodiments, the omega fatty acid is administered contemporaneously with the angioplasty, and with one or more antithrombotic agents.

These and other aspects of this invention will become evident upon reference to the following detailed description. To this end, all references identified herein are incorporated by reference in their entirety.

### DETAILED DESCRIPTION OF THE INVENTION

As mentioned above, this invention is directed to methods and compositions for treating a cardiovascular condition by intravascular administration of an omega fatty acid to a patient in need thereof.

As used herein, the term “cardiovascular condition” means an occlusive vascular condition that presents an occlusive vascular site that adversely affects blood flow in the cardiovascular system of a patient. The fundamental pathological feature of such conditions is the abnormal accumulation of cells within the vascular intimal space, resulting in neointimal lesion formation (thickening) produced by alterations in the homeostatic balance between cell growth and cell death. As mentioned above, representative cardiovascular conditions include (but are not limited to) coronary artery disease, myocardial infarction, cerebrovascular disease, stroke, peripheral vascular disease, and atherosclerosis or thrombosis of arteries or veins supplying any organ system. Thrombosis or restenosis can also occur in man-made grafts or stents, or in association with other therapeutic interventions such as angioplasty.

More specifically, occlusive vascular conditions are characterized by an occlusive vascular site having an abnormal accumulation of vascular smooth muscle cells, inflammatory cells, and extracellular matrix proteins within the intimal space between the endothelial lining and the medial layer (commonly referred to as atheromatous plaque). Current treatments are directed at either reducing the risk factors that promote occlusive vascular conditions, such as lowering cholesterol (LDL/cholesterol), or enhancing blood flow by interventions such as balloon angioplasty (with or without stent placement) or surgical revascularization (bypass). As mentioned above, angioplasty is a procedure in which a balloon is inserted into the vessel and then inflated to dilate the area of narrowing. In 30-50% of the cases, the initial increase in lumen dimensions is followed by a re-narrowing or restenosis of the vessel over a period of time ranging from 3-6 months. Restenosis is a complex process, resulting from cellular hyperproliferation within the neointima, the organization of thrombi within the vessel wall, and the process of vascular remodeling or shrinkage of the overall vessel dimensions.

In addition to angioplasty, bypass surgery with synthetic or vein grafts is a standard surgical approach to treat occlusive cardiovascular conditions. Vein grafts are essentially conduits that restore normal tissue blood flow by circumventing the occlusive arterial lesion. However, about 60% of such grafts occlude within 5-10 years because of neointimal hyperplasia and accelerated atherosclerosis within the graft. More recently, a process combining balloon angioplasty with intravascular stenting, using a cylindrical strut that expands the lumen, has been employed (Wickelgren, I., Science 272(3):668-670, 1996). When combined with antiproliferative and anticoagulant therapy, this process appears to be an effective therapy for treating occlusive cardiovascular conditions. Unfortunately, risks and costs associated with anticoagulant therapy using agents such as heparin and warfarin can be very high.

In the practice of this invention, treatment of an occlusive cardiovascular condition is accomplished by local intravascular administration of an omega fatty acid to a patient in need thereof at or near the site of occlusion. Accordingly, methods of this invention include local intravascular administration of an effective amount of a composition containing an omega fatty acid. Suitable compositions preferably include one or more carriers or diluents suitable for intravascular administration.

As used herein, the terms “treat” and “prevent” are intended to include the administration of an omega fatty acid to a patient for purposes which can include prophylaxis, amelioration, prevention or cure of a cardiovascular condition. Such treatment or prevention need not necessarily completely ameliorate the disease, and may be used in conjunction with other techniques now known or subsequently developed by those skilled in the art. In the case of many cardiovascular conditions, effective treatment or prevention includes delaying onset of the condition itself, or preventing the reoccurrence of the same. For example, in the case of restenosis, treatment includes delaying the onset of the same, while with regard to myocardial infarction preventing recurrence is desired.

Furthermore, the term “local intravascular administration” means that the omega fatty acid is delivered directly within the vascular lumen at or in close proximity to the site or sites of occlusion. In a preferred embodiment, local intravascular administration is employed. In the case of angioplasty, for example, administration is preferably accomplished by injecting the omega fatty acid into the vessel at the site of the angioplasty.

The omega fatty acids of this invention are a specific class or subset of fatty acids. In general, fatty acids are a class of organic compounds that are characterized by a long hydrocarbon chain terminating with a carboxylic acid group. Fatty acids have a carboxyl end and a methyl (i.e., “omega”) end. Omega-3 fatty acids are a family of unsaturated fatty acids where the unsaturated carbon most distant from the carboxyl group is the third carbon from the methyl terminus. Similarly, omega-6 fatty acids are a family of unsaturated fatty acids where the unsaturated carbon most distant from the carboxyl group is the sixth carbon from the methyl terminus.

The omega-3 fatty acids of this invention have the following general formula:

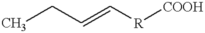

where R is a saturated or unsaturated, substituted or unsubstituted, branched or straight chain alkyl having from 1 to 20 carbon atoms. Preferably, R is an unsaturated straight chain alkyl having from 13 to 17 carbon atoms (i.e., an omega-3 fatty acid having from 18 to 22 total carbon atoms), and containing 2 to 6 carbon-carbon double bonds. In a particularly preferred embodiment, the omega-3 fatty acids of this invention contain 20 carbon atoms with 5 carbon-carbon double bonds, or 22 total carbon atoms with 6 total carbon-carbon double bonds, including (but not limited to) docosahexaenoic acid (DHA) and eicosapentaenoic acid (EPA):

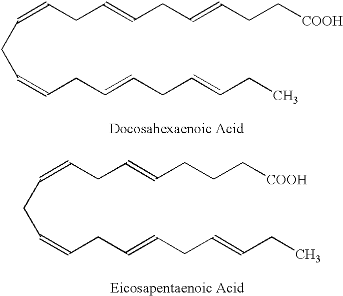

Similarly, the omega-6 fatty acids of this invention have the following general structure:

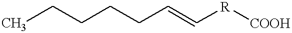

where R is a saturated or unsaturated, substituted or unsubstituted, branched or straight chain alkyl group having from 1 to 20 carbon atoms. Preferably, R is an unsaturated straight chain alkyl having from 10 to 14 carbon atoms (i.e., an omega-6 fatty acid having from 18 to 22 total carbon atoms), and containing from 2 to 6 carbon-carbon double bonds. In a preferred embodiment, the omega-6 fatty acids of the present invention contain 18 carbon atoms with 3 carbon-carbon double bonds, or 20 carbon atoms with 4 carbon-carbon double bonds, including (but not limited to) gamma-linolenic acid (“GLA”) and dihomo-gamma-linolenic acid (“DHGLA”):

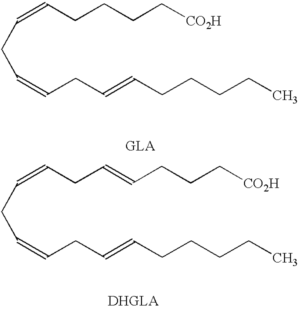

The omega fatty acid of the present invention may comprise an omega-3 fatty acid, an omega-6 fatty acid, a mixture of two or more omega-3 fatty acids, a mixture of two or more omega-6 fatty acids, or a mixture of one or more omega-3 and one or more omega-6 fatty acids.

Intravascular administration of an omega fatty acid is preferably accomplished by formulating the omega fatty acid within a composition comprising the omega fatty acid and one or more pharmaceutically acceptable carriers or diluents. Suitable compositions may contain a single omega fatty or a mixture of the same. Acceptable carriers or diluents are known in the art and include, for example, saline and sterile water. One skilled in this art may further formulate the omega fatty acid in an appropriate manner, and in accordance with accepted practices, such as those disclosed in Remington's Pharmaceutical Sciences, Gennaro (ed.), Mack Publishing Co., Easton, Pa., 1990. For example, a colloidal dispersion may be formed which contains the omega fatty acid by use of a suitable dispersion agent such as sodium desoxycholate. Furthermore, the omega fatty acid may be packaged within a liposomal or other microencapsulation delivery system. The omega fatty acid is present in the composition in an amount sufficient to treat or prevent the cardiovascular condition of the patient. Typically, representative compositions may contain the omega fatty acid in an amount ranging from 1% to 90% by weight (based on the total weight of the composition), more preferably from 10% to 80% by weight, and most preferably from 20% to 60% by weight.

Intravascular administration of a composition of the present invention provides advantages over other modes of administration. In particular, intravascular administration provides for rapid and increased delivery of the omega fatty acid to the treatment site. The method of administration is therefore significantly more selective than oral administration and, as a result, smaller dosages of the omega fatty acid are required. Also, the disadvantages associated with oral administration of omega fatty acids are substantially reduced.

Oral administration of fatty acids is generally associated with several drawbacks, including gastrointestinal absorption, metabolism and subsequent systemic distribution. These processes can substantially reduce the bioavailability and target organ specificity of orally administered fatty acids. To compensate, large amounts of omega fatty acids, typically in the range of 1-6 g/day (see, e.g., Endres, S. et al., Eur. J. Clin. Invest. 25:629-638, 1995), have been ingested over extended periods of time to achieve delivery of sufficient quantities to, for example, become significantly incorporated into serum phospholipids and cell membranes.

Furthermore, due to the relatively large doses required by oral delivery, the potential for side-effects is greater. Omega fatty acids affect a diverse set of biological processes reflecting, among other activities, their lipid-lowering, antihypertensive, anti-inflammatory, and antithrombotic actions (Engler M. B. et al., J. Cardiovasc. Nurs. 8(3):53-67, 1994; De Caterina R. et al., Arterioscler. Thromb. 14(11):1829-36, 1994). In the context of using systemic distribution to achieve results at a specific site, this pleotropic activity profile creates the potential for side-effects. For example, omega-3 fatty acids are known to interfere with normal platelet function (aggregation and growth factor production), and oral administration generally results in increased capillary fragility and danger of bleeding (Rogers et al., Atherosclerosis 63:137-143, 1987). Immune reactivity is generally reduced by omega-3 fatty acids and, while this may be beneficial in some conditions, could be detrimental under other circumstances. Furthermore, in addition to the bioavailability and target organ specificity aspects pertaining to orally administered agents, fundamental differences in biological response are known to be associated with route of delivery.

Although not intending to be limited to the following theory, it is believed that the compositions of the present invention effectively treat and/or prevent cardiovascular conditions by modulating many of the biological processes that promote atherosclerosis, coronary artery disease, and myocardial infarction. For example, Leaf et al. (supra) describes atherosclerosis as a dysfunctional state of the arterial endothelial cells. This results from stresses including hemodynamic, elevated LDL cholesterol, virus infections, toxins, trauma, etc. The first visible effect of dysfunction is the adhesion of circulating monocytes to the affected endothelial cells. Further changes involve the deposition of macrophages in the underlying intima, the release of free radicals by macrophages, and the oxidation of lipids. The oxidized LDL particles are then recognized by scavenger receptors. Subsequent developments include inflammatory and immunological response with migration of cells, platelets, lymphocytes, cytokines, and other cellular messengers.

The omega-3 fatty acids have important antiatheromatous actions because of their effect on platelets and the cycloxygenase system. Thromboxane A₂ has highly potent vasoconstricting and platelet-aggregating effects. Prostacyclin (prostaglandin I₂) is the principle cycloxygenase product in endothelial cells and its actions counterbalance those of thromboxane, since it is vasodilatory and antiaggregatory. The omega-3 fatty acids compete with arachadonic acid and inhibit the synthesis of arachadonic acid from linolenic acid. They compete with arachadonic acid vis-a-vis incorporation in membrane phospholipids, thereby reducing plasma and cellular levels of arachadonic acid. Hence, with less arachidonic acid and more omega fatty acid, there is less production of thromboxane AZ and increased production of prostacyclin. The net result is a change in the hemodynamic balance toward a vasodilatory state with less platelet aggregation.

While the mechanism of the antiatheromatous effects of omega-3 fatty acids is uncertain, Leaf and Weber (NEJM 318(9):549-57, 1988) postulate that these compounds increase the formation of prostacyclin, thereby decreasing the thrombogenic potential of endothelial surfaces. These authors further postulate that these compounds, by blocking the production of thromboxane, allow fewer or smaller platelet thrombi to form. Fewer platelets causes a reduction in the amount of platelet-derived growth factor (PDGF) that is released, which in turn decreases the amount of intimal hyperplasia of the blood vessel as well as the formation of fewer foam cells. Decreases in leukotriene B₄ decreases the inflammatory response. All of these factors inhibit the formation of atheromatous plaques. Other investigators have reported that omega-3 fatty acids reduce blood viscosity, plasma levels of fibrinogen and beta-thromboglobulins. They also increase capillary blood flow and red cell membrane fluidity, presumably leading to better tissue oxygenation (Semplicini and Valle, Pharmacology and Therapeutics 61(3):385-97, 1994).

The migration of inflammatory cells to the wall of the blood vessel promotes atheromata formation, a process inhibited by omega-3 fatty acids. They also competitively inhibit leukotriene production and shift leukotrienes to less inflammatory forms. Like prostaglandins, leukotrienes also are active mediators of the inflammatory response. Omega-3 fatty acids have been shown to reduce the levels of leukotrienes as well as shunting the formation of leukotriene B₄ to the less inflammatory leukotriene B₅. The net result is decreased chemotaxis (white blood cell mobilization), decreased cellular traffic, and overall dampening down of the immune response.

More recent cardiovascular research has focused on the immune mechanisms underlying cardiac disease, suggesting that autoimmune factors may play a significant role in coronary artery disease, myocarditis and cardiomyopathies (Lange and Schreiner, NEJM 330(16):1129-35, 1994; Dahlen, G. et al., Atherosclerosis 114(2):165-74, 1995). Omega-3 fatty acids dampen the immune response, and this down regulation results in cardioprotection.

Further studies on the role of autoimmune mechanisms and coronary artery disease have demonstrated increased anticardiolipin (ACL) antibodies in patients with restenosis after PTCA. The investigators compared patients with and without restenosis following PTCA. There were no differences demonstrated between the two groups with respect to medical history, laboratory findings, or vascular risk factors. In patients with restenosis acL were more often increased. It has been suggested that an autoimmune mechanism might play a role in restenosis (Eber et al., American Journal of Cardiology 69(16):1255-58, 1992). Omega-3 fatty acids, with their role as modulators of the immune response, as well as their antithrombotic action, could exert a potentially beneficial effect in blocking this response.

In addition, it is believed that omega-3 fatty acids reduce thrombosis, intimal hyperplasia, and atherosclerosis by enhancing the release of endothelium-dependent relaxation factor (EDRF), thereby decreasing platelet aggregation and adherence to endothelium. This leads to decreased release of thromboxane A₂ and platelet derived growth factor. Omega-3 fatty acids also inhibit endothelin, a potent vasoconstrictor, as well as the pro-coagulant and endothelial binding effects of the cytokines IL-1 and TNF. The net effect of the above actions is decreased vasoconstriction, platelet and leukocyte adherence, and endothelial cell proliferation.

Evidence from both human and nonhuman studies have demonstrated that fatty acids in general, in addition to omega-3 fatty acids, are safe and effective immunomodulatory and antithrombotic agents. Plant seed oils, particularly those derived from the evening primrose and borage plants, are rich in the omega-6 fatty and gamma-linolenic acid (GLA). This is converted to dihomogamma-linolenic acid (DGLA), the fatty acid precursor to PGE1 and TXA1 via the cyclooxygenase pathway and (150H)DGLA and LTC3 via the lipoxygenase pathway. Similar to the situation with the cicosanoid products of the fish oils, these eicosanoids have been shown to have anti-inflammatory, immunomodulatory, and antithrombotic actions compared to the pro-inflammatory and thrombotic action of their counterparts derived from arachidonic acid, PGE2, TXA2, and LTB4. In particular, PGE1 has been shown to inhibit aggregation of human platelets in vitro and relaxes muscle arterioles, both important antithrombotic and vasodilatory actions similar to the action of the omega-3 metabolites.

While there is discussion in the literature on the effect of long-term dietary supplementation with polyunsaturated fatty acids on the modulation of arteriosclerosis, clinical efficacy has yet to be established. Moreover, no one has considered the role of the direct, intravascular administration of these compounds for treating or preventing, for example, atheromatous plaque formation and/or restenosis of vessels in the acute clinical setting. Direct intravascular application of an omega fatty acid at the time of angioplasty as disclosed herein would be effective for treating restenosis with or without antithrombotic agents such as TPA and streptokinase. The beneficial effects include decreased morbidity and mortality, as well as great savings in health care costs.

The following examples are provided for purposes of illustration, not by way of limitation.

### EXAMPLES

### Example 1

### Representative Compositions

The following Formulations are representative compositions suitable for intravascular administration of an omega fatty acid to a patient in need thereof.

| Formulation I | % by weight |
|---|---|
| Eicosapentaenoic acid | 30-50 |
| Docosahexaenoic acid | 5-10 |
| Sodium desoxycholate | 0-8 |
| Saline and other inert ingredients | 32-65 |

| Formulation II | % by weight |
|---|---|
| Gammalindenic acid and/or | 30-50 |
| dihomogammalinolenic acid | |
| Sodium desoxycholate | 0-8 |
| Saline and other inert ingredients | 42-70 |

| Formulation III | % by weight |
|---|---|
| Gammalindenic acid and/or | 15-25 |
| dihomogammalinolenic acid | |
| Eicosapentaenoic acid | 15-25 |
| Docosahexaenoic acid | 5-10 |
| Sodium desoxycholate | 0-8 |
| Saline and other inert ingredients | 32-65 |

### Example 2

### Administration of a Representative Composition to Patients in Need Thereof

Case 1

A 69 year old male presents with chest pain and an acute MI is diagnosed. In the coronary care unit, TPA is administered: 6 mg bolus followed by 54 mg the first hour and 20 mg each of the subsequent two hours. There is no change in the electrocardiogram (EKG) pattern; chest pain and ventricular arrhythmias persist. Cardiac catheterization reveals complete distal left anterior descending (LAD) occlusion. Percutaneous transluminal coronary angioplasty (PTCA) is attempted but cannot be accomplished. Intra-coronary infusion of a composition containing an omega fatty acid follows and results in thrombus dissolution.

Case 2

A 68 year old white male develops chest pain while exercising on his home treadmill. His wife calls the Medics who arrive within four minutes. EKG reveals signs of an acute MI. He is hypotensive, short of breath, and has a rapid heart rate. After transport to the coronary care unit, he is seen by a cardiologist, who recommends PTCA. At coronary catheterization, complete blockage of the left anterior descending artery is noted. Angioplasty is attempted but substantially fails due to thrombosis with restenosis. A composition containing an omega fatty acid is immediately infused through the coronary catheter. Thrombotic occlusion improves and coronary blood flow is reestablished. Chest pain resolves and blood pressure normalizes. A follow-up angiogram reveals normal patency after 12 months.

Case 3

A 75 year old male presents with increasingly frequent episodes of chest pain six months after PTCA of a 90% occluded circumflex artery. Cardiac catheterization reveals restenosis at the same site. A composition containing an omega fatty acid is infused via catheter, resolving the stenotic lesion.

Case 4

A 63 year old male has crescendo angina. He is found to have severe three-vessel coronary disease at cardiac catheterization, and coronary artery bypass grafting is recommended. At completion of surgical grafting, the vascular surgeon infuses a composition containing an omega fatty acid into the coronary arteries in order to reduce the incidence of post-surgical thrombosis.

Case 5

A 63 year old male is admitted to the hospital because of unstable angina. A coronary artery catheterization reveals severe narrowing of the left anterior descending artery. Balloon angioplasty is attempted, but the stenotic lesion cannot be dilated successfully. A stent is then placed in the LAD that successfully dilates the coronary artery. A composition containing an omega fatty acid is infused with the stent to preserve patency. The patient has an uneventful recovery and leaves the hospital pain free. At 12 months follow-up the stent is still functioning well, and the patient remains asymptomatic.

Case 6

A 59 year old male is readmitted to the hospital for chest pain, the patient having undergone balloon angioplasty three months earlier. During admission, catheterization reveals restenosis of the artery that had previously undergone angioplasty. On coronary catheterization the lesion is restenosed. Due to the failure of angioplasty it is decided that a stent needs to be placed. After the stent is placed, the patient receives an infusion in the stented coronary artery of a composition containing an omega fatty acid to preserve patency and avoid thrombosis.

Case 7

A 64 year old female with a long history of smoking and peripheral vascular disease is admitted to the hospital with severe pain in the right lower extremity. The pulses in the patient's right distal lower extremity are absent. The right foot is cold. Arteriography reveals stenosis at the right iliac artery. A radiologist places a stent in the iliac artery bypassing the point of obstruction. A compound containing an omega fatty acid is then infused to preserve patency and avoid thrombosis. Blood flow is returned to the right lower extremity. The patient's symptoms resolve, pulses are restored and the patient leaves the hospital uneventfully.

Case 8

A 59 year old male with exertional angina undergoes routine PTCA for a 90% stenosis of the left anterior descending coronary artery. The procedure goes well without complication, and normal blood flow is reestablished. To prevent restenosis, prior to removing the angioplasty catheter, a composition containing both an omega-3 and omega-6 fatty acid is administered via the catheter. One year follow-up demonstrates continued patency of the vessel.

Case 9

A 42 year old female with lupus presents with sudden onset pain and swelling in her posterior calf. Manifestations of lupus include arthritis, rash, interstitial lung disease, and significant leucopenia and thrombocytopenia. Her white blood count is 2.5 and platelet count 35,000, both quite low. She has had two previous miscarriages and is known to be anticardiolipin antibody positive, which predisposes her to thrombosis. She has been treated with prednisone, but not aspirin or coumadin because of her low platelet count. A deep vein thrombosis of the leg is suspected because of calf pain and confirmed by venous Doppler study. To treat this, avoid pulmonary embolus, and avoid the bleeding complications that could ensue from heparin use in the face of thrombocytopenia, her managing physicians employ omega-3 and omega-6 fatty acid treatment. The radiologist infuses a composition containing the omega-3 and omega-6 fatty acids via a lower extremity venogram. The thrombosis is cleared and there is no evidence of either bleeding complications or blood clot to the lungs.

Case 10

A 53 year old female with chronic diabetes mellitus and renal failure is hospitalized because of confusion. Renal function has precipitously declined with creatinine 6.5 and Bun of 110. Her nephrologist recommends dialysis. The vascular surgeon emergently establishes a vascular access graft. However prior to initiation of dialysis the graft thromboses. Because the patient is discovered to have an actively bleeding ulcer heparin is contraindicated. A composition containing an omega fatty acid is infused, clearing the thrombosis and providing patency of the graft for hemodialysis.

Case 11

A 67 year old female with chronic renal failure undergoes hemodialysis on a regular basis. She is now on her third A-V graft, the first two having occluded after a fairly short time. As part of her routine dialysis procedure, the patient now receives two bolus injections of a mixture containing both omega-3 and omega-6 fatty acids, one as soon as the graft is accessed, the other immediately before completion of the dialysis procedure. The current graft remains functional for over two years.

From the foregoing, it will be appreciated that, although specific embodiments of the invention have been described herein for purposes of illustration, various modifications may be made without departing from the spirit and scope of the invention. Accordingly, the invention is not limited except as by the appended claims.

What is claimed is:

1. A method for treating or preventing a cardiovascular condition presenting an occlusive vascular site that adversely affects blood flow in the cardiovascular system of a patient, comprising intravascularly administering to the occlusive vascular site an effective amount of an omega fatty acid.

2. The method of claim 1 wherein the cardiovascular condition is selected from coronary artery disease, myocardial infarction, cerebral vascular disease, stroke, peripheral vascular disease and atherosclerosis.

3. The method of claim 1 wherein the occlusive vascular site is due to thrombosis or restenosis.

4. The method of claim 3 wherein thrombosis or restenosis occurs in a graft or stent.

5. The method of claim 4 wherein the graft is a dialysis access graft.

6. The method of claim 3 wherein thrombosis or restenosis occurs following angioplasty.

7. The method of claim 1 wherein the omega fatty acid is an omega-3 fatty acid.

8. The method of claim 7 wherein the omega-3 fatty acid has the following structure: 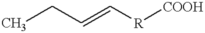
wherein R is a saturated or unsaturated, substituted or unsubstituted, branched or straight chain alkyl having from 1 to 20 carbon atoms.

9. The method of claim 7 wherein the omega-3 fatty acid is eicosapentaenoic acid.

10. The method of claim 7 wherein the omega-3 fatty acid is docosahexaenoic acid.

11. The method of claim 1 wherein the omega fatty acid is an omega-6 fatty acid.

12. The method of claim 11 wherein the omega-6 fatty acid has the following structure:

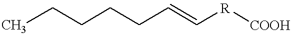

13. The method of claim 11 wherein the omega-6 fatty acid is gamma-linolenic acid.

14. The method of claim 11 wherein the omega-6 fatty acid is dihomogamma-linolenic acid.

15. The method of claim 1 wherein the omega fatty acid is a mixture of an omega-3 fatty acid and an omega-6 fatty acid.

16. The method of claim 1 wherein the omega fatty acid is administered as a composition comprising the omega fatty acid and a pharmaceutically acceptable carrier or diluent.

17. A method of preventing restenosis following angioplasty, comprising locally and intravascularly administering to a patient in need thereof an omega fatty acid at the site of the angioplasty.

18. The method of claim 17 wherein the omega fatty acid is administered contemporaneously with the angioplasty.

19. The method of claim 17 wherein the omega fatty acid is administered in combination with an antithrombotic agent.

20. A method for preventing thrombosis or restenosis of a dialysis vascular access graft, comprising locally and intravascularly administering to a patient in need thereof an omega fatty acid at the site of the graft.
